(19) United States
(12) Patent Application Publication
(10) Pub. No.: US 2009/0012607 A1
(43) Pub. Date: Jan. 8, 2009

(54) METHOD FOR THE PREPARATION OF TUBE-TYPE POROUS BIODEGRADABLE SCAFFOLD HAVING DOUBLE-LAYERED STRUCTURE FOR VASCULAR GRAFT

(75) Inventors: Sang-Heon KIM, Seoul (KR); Soo Hyun KIM, Seoul (KR); Eunna CHUNG, Seoul (KR)

Correspondence Address: H.C. PARK & ASSOCIATES, PLC, 8500 LEESBURG PIKE, SUITE 7500, VIENNA, VA, 22182, US

(73) Assignee: KOREA INSTITUTE OF SCIENCE AND TECHNOLOGY, Seoul (KR)

(21) Appl. No.: 11/952,759

(22) Filed: Dec. 7, 2007

(30) Foreign Application Priority Data
Jul. 6, 2007 (KR) 10-2007-0067907

(51) Int. Cl. A61F 2/82; B29C 70/32

(52) U.S. Cl. 623/1.46; 264/46.4

(57) ABSTRACT

Disclosed herein are a tube-type porous scaffold having a double-layered structure for use as an artificial vascular graft and a preparation method thereof. The method comprises (1) dissolving a biodegradable polymer in an organic solvent and mixing the polymer with a porogen so as to provide a polymer/porogen mixture; (2) coating a cylindrical shaft with the polymer/porogen mixture so as to form an inner porous coating layer; (3) preparing a biodegradable polymer gel by dissolving a biodegradable polymer in an organic solvent; (4) spinning down the biodegradable polymer gel in a non-solvent coagulation bath in which the cylindrical shaft having the inner porous coating layer, obtained at step (2), is immersed and rotated to form gel-phase fibers and allowing the gel-phase fibers to wind around the inner porous coating layer of the rotating shaft so as to form an outer polymer fibrous layer; and (5) separating the double-layered porous scaffold, formed on the shaft, from the shaft and removing the organic solvent and the porogen from the scaffold. Since the porous scaffold has a double-layered structure consisting of an inner porous coating layer containing micropores and a gel-phase outer polymer fibrous layer, it has high pore interconnectivity and mechanical strength, which effectively prevents the leakage of blood, and has high cell seeding and proliferation efficiencies, thereby being useful as a tissue-engineered artificial vascular graft.

### BACKGROUND OF THE INVENTION

1. Field of the Invention

The present invention relates to a method of preparing a double-layered porous scaffold having different pore sizes by coating a cylindrical shaft with a mixture of a biodegradable polymer and a porogen so as to provide an inner porous coating layer, and directly spinning down a biodegradable polymer gel into a non-solvent coagulation bath, in which the cylindrical shaft is immersed and rotated to form gel-phase polymer fibers, and allowing the gel-phase polymer fibers to wind around the inner porous coating layer of the rotating shaft so as to provide an outer layer, the inner and outer layers being attached to each other. The present invention is also concerned with a porous scaffold having a double-layered structure for use as an artificial vascular graft, which is prepared using the method.

2. Description of the Related Art

Most early approaches to the tissue engineering of blood vessels focused on the use of natural polymers, such as collagen, or biodegradable synthetic polymers, such as PGA, which are formed in a tubular shape, seeded with smooth muscle cells or endothelial cells, constituting the vessel tissue, cultivated for a given period of time in vitro so as to have a certain degree of mechanical strength, and then implanted into the body. Recent advances in stem cells have led to the implantation of a tubular porous support matrix seeded with stem cells without in vitro cultivation (Narutoshi Hibino et. al., J. Thoracic and Cardiovascular Surgery 129: 1064-1670, 2005). Since this approach involves implanting the porous support immediately after being seeded with stem cells without in vitro cultivation, the scaffold itself should have sufficient mechanical strength to withstand in vivo forces. In other words, since an artificial blood vessel is an artificial organ that substitutes a damaged vessel in the body and restores blood flow, the vessel construct should have burst strength that is high enough to withstand the blood pressure in the body, and should be made of a highly elastic material that is able to expand and contract with the beating heart, like natural vessels. As well, blood leakage, which may occur early after implantation, is a critical factor that influences the success of implantation of artificial blood vessels.

Conventional prosthetic vascular grafts made of expanded polytetrafluoroethylene (ePTFE) and polyethylene terephthalate (PET) satisfy the above requirements, but cannot be used in practice as tissue-engineered artificial blood vessels for inducing regeneration of the body blood tissue because the materials are non-degradable in the body. Currently available artificial blood vessels, achieved through tissue engineering technologies using stem cells, have been limited in the clinical application thereof to the vena cava and the pulmonary artery, which are at relatively low pressure. No tissue-engineered artificial blood vessels that can endure the high pressure environment of arterial flow have been developed.

A tubular scaffold is generally constructed by winding a non-woven mesh of polyglycolic acid (PGA) fibers or a woven mesh of poly(L-lactic acid) (PLLA) fibers, which is a biodegradable material, around a cylindrical shaft and sewing the polymer scaffold into a tubular shape using a surgical suture, or by immersing a PGA or PLLA fiber mesh in a solution of a polymer having different dissolution property, such as poly(L-lactic acid-co-caprolactone) (PLCL), and lyophilizing (freeze-drying) the polymer mesh. Freeze-drying using PLCL is employed to form pores in the polymer scaffold, but the use of PGA or PLLA has problems in that PGA or PLLA has considerably lower elasticity than that of PLCL and in that its degradation rate is difficult to control. Also, the porous structure limits the use of the scaffold as a vascular graft with no blood leakage under high blood pressure. An artificial blood vessel construct made of PLCL alone, which is fabricated mainly through freeze-drying, casting, extrusion, and the like, has low cell seeding efficiency and weak mechanical strength.

Thus, there remains a need for the development of a porous scaffold as a tissue-engineered artificial blood vessel having high elasticity and mechanical strength.

In this regard, the inventors of this application conducted intensive and thorough research in order to overcome the problems encountered in the prior art. The research resulted in the development of a method of preparing a double-layered porous scaffold having different pore sizes by coating a cylindrical shaft with a mixture of a biodegradable polymer and a porogen so as to provide an inner porous coating layer, and directly spinning down a biodegradable polymer gel into a non-solvent coagulation bath, in which the cylindrical shaft is immersed and rotated to form gel-phase polymer fibers and winding the gel-phase polymer fibers around the inner porous coating layer of the rotating shaft so as to provide an outer layer, the inner and outer layers being attached to each other. The porous scaffold prepared according to the method was found, owing to its double-layered tubular structure, to have high interconnectivity between pores and high mechanical strength, which may effectively prevent blood leakage, as well as high cell seeding and proliferation efficiencies, which may allow the use as an artificial vascular graft, thereby leading to the present invention.

### SUMMARY OF THE INVENTION

Accordingly, the present invention aims to provide a porous scaffold for use as an artificial vascular graft and a preparation method thereof, the scaffold having good pore interconnectivity, mechanical strength and cell seeding and proliferation efficiencies and being capable of preventing the leakage of blood at high pressures, such as in arteries.

In order to accomplish the above objects, the present invention provides a method of preparing a tube-type porous scaffold having a double-layered structure comprising the steps of (1) dissolving a biodegradable polymer in an organic solvent and mixing the polymer with a porogen so as to provide a polymer/porogen mixture; (2) coating a cylindrical shaft with the polymer/porogen mixture so as to form an inner porous coating layer; (3) preparing a biodegradable polymer gel by dissolving a biodegradable polymer in an organic solvent; (4) spinning down the biodegradable polymer gel in a non-solvent coagulation bath in which the cylindrical shaft having the inner porous coating layer, obtained at step (2), is immersed and rotated to form gel-phase fibers and allowing for the gel-phase fibers to wind around the inner porous coating layer of the rotating shaft so as to form an outer polymer fibrous layer; and (5) separating the double-layered porous scaffold, formed on the shaft, from the shaft and removing the organic solvent and the porogen from the scaffold.

In addition, the present invention provides a tube-type porous scaffold having a double-layered structure for use as a biodegradable and biocompatible artificial vascular graft.

Unlike conventional methods of preparing single-layered porous scaffolds fabricated through gel spinning molding, the present method is featured by first forming an inner porous coating layer containing micropores, which prevent the leakage of blood, winding gel-phase polymer fibers around the inner layer so as to create an outer layer, and allowing for the inner porous coating layer and the outer polymer fibrous layer to attach to each other, thereby fabricating a tubular porous scaffold having a double-layered structure, each layer having a different pore size.

In general, scaffolds fabricated using a gel spinning molding technique have large pore sizes and very high interconnectivity between pores, which result in the leakage of erythrocytes under pressure similar to physiological blood pressure. In order to overcome these drawbacks of gel spinning molding, the inventors of this application developed a method of constructing a porous scaffold in a double-layered structure by primarily coating a cylindrical shaft, used in gel spinning molding, with a mixture of a biodegradable polymer and a porogen so as to form an inner porous coating layer containing micropores, and then forming a gel-phase outer polymer fibrous layer on the inner layer and allowing the outer layer to attach to the inner layer.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a schematic diagram showing the process of preparing a tube-type porous scaffold having a double-layered structure for use as an artificial vascular graft according to the present invention;

FIG. 2 is a schematic representation of a gel spinning device for spinning a biodegradable polymer gel onto a shaft coated with a polymer/porogen mixture according to the present invention;

FIG. 3a is an SEM micrograph of the outer surface of a double-layered porous poly(L-lactic acid-co-caprolactone) (50:50) scaffold (PLCL to NaCl=1:1) prepared in Example 1 according to the present invention;

FIG. 3b is a SEM micrograph of the cross section of a double-layered porous PLCL (50:50) scaffold (PLCL to NaCl=1:1) prepared in Example 1 according to the present invention;

FIG. 3c is an SEM micrograph of the inner surface of a double-layered porous PLCL (50:50) scaffold (PLCL to NaCl=1:1) prepared in Example 1 according to the present invention;

FIG. 4a is an SEM micrograph of the cross section of a single-layered PLCL (50:50) scaffold prepared in Comparative Example 1 according to a conventional method;

FIG. 4b is an SEM micrograph of the inner surface of a single-layered PLCL (50:50) scaffold prepared in Comparative Example 1 according to a conventional method; and

FIG. 5 is an SEM micrograph of a double-layered porous PLCL (50:50) scaffold (PLCL to NaCl=9:1) prepared according to the present invention, which was seeded with bone marrow mononuclear cells, which were allowed to grow.

### DESCRIPTION OF THE PREFERRED EMBODIMENTS

At step (1) of the method, a biodegradable polymer is dissolved in an organic solvent and mixed with a porogen, which is dispersed in the solvent, in order to provide a polymer/porogen mixture for the cylindrical shaft coating.

The biodegradable polymer suitable for use in step (1) is an aliphatic polyester. Examples of aliphatic polyesters include poly(L-lactic acid) (PLLA), poly(D,L-lactic acid) (PDLLA), polyglycolic acid (PGA), polycaprolactone (PCL), polyhydroxyalkanoate, polydioxanone (PDS), and polytrimethylene carbonate. Also, a copolymer of the monomers may be used. Examples of copolymers include poly(lactic acid-co-glycolic acid) (PLGA), poly(L-lactic acid-co-caprolactone) (PLCL), poly(glycolic acid-co-caprolactone) (PGCL), and derivatives thereof. The biodegradable polymer may be used regardless of its molecular weight, but it is advantageous in the preparation of the porous scaffold according to the present invention to use a polymer having a molecular weight of greater than 5,000 Daltons, preferably ranging from 5,000 to 1,000,000 Daltons. In addition, the biodegradable polymer is dissolved in an organic solvent in an amount of 1% to 20% based on its weight to volume ratio (w/v).

The organic solvent used for dissolving the biodegradable polymer may include chloroform, methylene chloride, acetic acid, ethylacetate, dimethylcarbonate, and tetrahydrofuran.

The porogen, which is mixed with the biodegradable polymer solution, is employed to the formation of micropores when an inner porous coating layer is formed on a cylindrical shaft. The size and form of pores may be controlled by varying the size, kind and amount of the porogen. This is critical for preventing the leakage of blood from the porous scaffold.

The porogen useful in the present invention includes those commonly used for generating pores in the art. Examples of such porogens include, but are not limited to, sodium chloride, sodium bicarbonate, ammonium bicarbonate, paraffin and polyethylene glycol. The porogen is preferably mixed with the biodegradable polymer in the biodegradable polymer solution at a weight ratio of 9:1 to 1:2 (polymer:porogen). If the mixing ratio of the biodegradable polymer to the porogen exceeds 1:2, the number of pores may increase, causing the leakage problem. If the polymer to porogen ratio is lower than 9:1, nutrient supply and neovascularization may be inhibited after implantation. In addition, the porogen preferably has a diameter of less than 40 microns. If the diameter of the porogen exceeds 40 microns, larger pores may be formed, leading to the leakage of blood. However, it will be apparent to those skilled in art that the size, kind and amount of the porogen may vary depending on the form and size of desired pores.

At step (2) of the method, a cylindrical shaft is coated with the polymer/porogen mixture obtained at step (1) in order to form an inner porous coating layer containing micropores. The micropores play an important role in preventing the leakage of blood.

The formation of the inner porous coating layer on the cylindrical shaft may be achieved through extrusion, impregnation, electrospinning, freeze-drying, phase separation, particle leaching, gas foaming, hydrocarbon templating, melt molding, or the like, but the present invention is not limited thereto. In a preferred embodiment of the present invention, according to the impregnation method, the cylindrical shaft is impregnated in the polymer/porogen mixture at a state of being sufficiently immersed therein, and coating is carried out at 4° C. to 25° C. for 5 to 20 minutes so as to provide an inner porous coating layer. The inner porous coating layer preferably contains pores having a size of less than 40 microns.

At step (3) of the method, a biodegradable polymer is dissolved in an organic solvent to provide a biodegradable polymer gel. The kind and mixing ratio of the biodegradable polymer and the organic solvent suitable for use in step (3) are the same as described at step (1), except that the biodegradable polymer gel is not mixed with a porogen unlike the polymer solution of step (1), and that the concentration of the biodegradable polymer may vary depending on the type thereof, but the polymer is preferably present in a concentration ranging from 4 to 20 wt %. The concentration of the biodegradable polymer is a very critical factor in controlling the thickness of gel-phase polymer fibers formed through phase separation and the porosity and pore size of an outer layer, which is made out of the polymer fibers. Thus, when the concentration of the biodegradable polymer exceeds 20 wt %, the polymer gel is too viscous to be spun using a syringe. When the biodegradable polymer is present at lower than 4 wt %, the spun gel-phase fibers are prone to break, thus decreasing the strength of a fabricated scaffold.

At step (4) of the method, the cylindrical shaft having the inner porous coating layer, obtained at step (2), is immersed and rotated in a non-solvent coagulation bath. Then, the biodegradable polymer gel is spun down through a syringe into the non-solvent coagulation bath. The spun polymer gel undergoes phase separation into gel-phase fibers, which winds around the rotating shaft. At this time, the polymer fibers wind around the inner porous coating layer while forming an outer layer, which is attached to the inner layer, resulting in the fabrication of a double-layered porous scaffold. In the double-layered porous scaffold, the outer polymer fibrous layer has a pore size different from that of the inner porous coating layer. The outer layer contains pores that preferably have a size ranging from 10 to 500 microns, and the mean pore size is preferably 100 microns.

The non-solvent used in step (4) functions to coagulate the spun biodegradable polymer gel at a proper rate. It is preferable to employ a non-solvent, which is readily miscible with the organic solvent used for dissolving a biodegradable polymer at step (3) and thus allows the phase separation of the spun polymer gel into a gel phase at a proper rate.

The non-solvent suitable for use in the present invention may include water; alcohols, such as methanol, ethanol and butanol; hydrocarbons, such as hexane, heptane and cyclohexane; and mixtures thereof.

In addition, the coagulation rate of the spun biodegradable polymer in the non-solvent coagulation bath is a critical factor in the attachment of the gel-phase fibrous polymer, formed through phase separation, to the inner porous layer containing micropores coated onto the shaft. The attachment between the fibrous polymer and the inner porous layer should be suitably maintained in order to construct a porous scaffold having uniform pore size and good pore interconnectivity. The attachment is induced by the solvent remaining in the fibrous polymer gel. That is, when the fibrous polymer gel is wound around the inner porous coating layer to thus form an outer layer, the remaining solvent melts the inner polymer layer, leading to attachment between the inner and outer layers. The coagulation rate of the spun biodegradable polymer in the non-solvent coagulation bath may be controlled depending on the kind and stirring rate of the non-solvent solution. To achieve a desired coagulation rate of the biodegradable polymer, it is preferable to employ a solvent which is able to induce the attachment between the gel-phase polymer fibers and the attachment between the fibrous polymer and the inner porous coating layer. If the coagulation very rapidly occurs, the attachment may not be formed between the gel-phase polymer fibers or between the fibrous polymer and the inner porous coating layer. If the coagulation rate is very slow, the gel-phase fibrous polymer may not be produced, and pores rarely form. Taking into account the pore size and porosity of the outer layer, the polymer fibers, present in a gel phase through phase separation after being spun into the non-solvent coagulation bath, preferably have a diameter ranging from 50 to 150 microns.

At step (5) of the method, the double-layered porous scaffold fabricated on the cylindrical shaft at step (4) is separated from the shaft, and the organic solvent and the porogen are removed from the scaffold. The organic solvent is removed through drying under reduced pressure. The porogen is removed by dissolving the porous scaffold in a solution capable of dissolving the porogen.

The porous scaffold, prepared according to the method as described above, has a tubular double-layered structure in which an outer layer made of a spun fibrous polymer surrounds an inner porous coating layer containing micropores. The inner porous coating layer, containing micropores, functions to prevent the leakage of blood from the scaffold after implantation. The outer polymer fibrous layer increases the interconnectivity between pores and the mechanical strength of the scaffold so that it prevents the scaffold from bursting under the high pressure in the body.

Hence, the tube-type porous scaffold having a double-layered structure, fabricated according to the method, may be useful as a biodegradable and biocompatible tissue-engineered artificial blood vessel.

A better understanding of the present invention may be obtained through the following examples, which are set forth to illustrate, but are not to be construed as the limit of the present invention.

### Example 1

### Preparation of Tube-Type Porous Scaffolds Having a Double-Layered Structure

PLCL (50:50 composition ratio of monomers) having a molecular weight of 450,000 Da was dissolved in chloroform at a concentration of 7.0% (w/v). Sodium chloride less than 20 microns in diameter was separated through sieving, and was mixed with the PLCL solution at PLCL to NaCl ratios of 1:1, 2:1 and 9:1. A cylindrical shaft 6.5 mm in diameter was immersed in the PLCL/NaCl mixture to a depth of about 10 cm, and was impregnated at 25° C. for 15 min, thereby forming an inner porous coating layer containing micropores on the surface of the cylindrical shaft.

The cylindrical shaft having the inner porous coating layer was immersed in a coagulation bath containing methanol and rotated at 300 rpm. PLCL, having the same molecular weight as that used for forming the inner layer, was dissolved in chloroform at a concentration of 7.5% (w/v), poured into a syringe of a gel spinning device, and spun down through the syringe using a syringe pump into the coagulation bath. The spun biodegradable polymer gel underwent phase separation into gel-phase polymer fibers. The gel-phase polymer fibers were allowed to wind around the inner porous coating layer of the cylindrical shaft rotating in the coagulation bath. At this time, the attachment between the inner porous coating layer and the outer polymer fibrous layer was induced by the solvent remaining in the polymer fibrous gel, which was wound around the inner layer, thereby fabricating a porous scaffold having a double-layered structure. Then, the cylindrical shaft was dried in a vacuum dryer to separate the double-layered porous scaffold from the shaft.

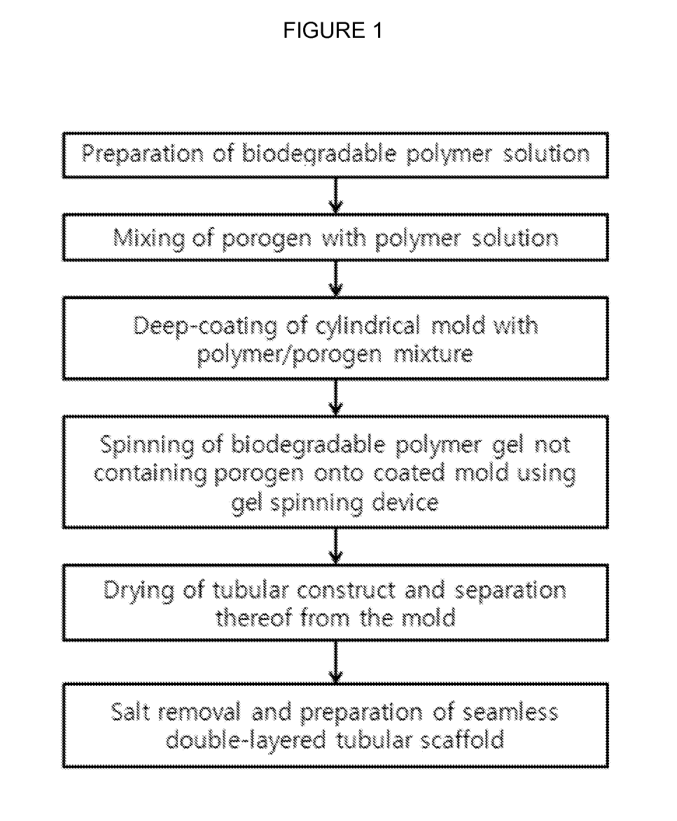

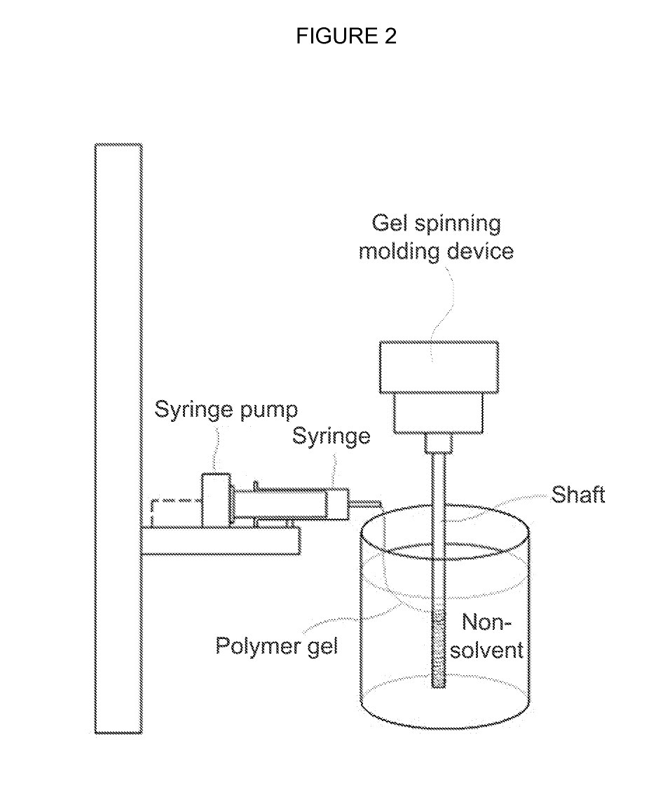

FIG. 1 is a schematic diagram showing the preparation of a tube-type porous scaffold having a double-layered structure for use as an artificial vascular graft according to the above procedure. FIG. 2 is a schematic representation of a gel spinning device for spinning a biodegradable polymer gel into a non-solvent coagulation bath according to the present invention.

The porous scaffold prepared according to the above procedure had a double-layered tubular structure. The inner porous coating layer and the outer polymer fibrous layer were attached to each other and had different pore sizes. In detail, the tube-type porous scaffold had an inner diameter of 6.5 mm and a thickness of 1.0 mm. The fibers, constituting the outer layer of the scaffold, were individually 30 to 100 microns in diameter. Also, the inner porous coating layer had a pore size of 15 microns, and the outer polymer fibrous layer had a pore size ranging from 50 to 150 microns. Further, the porosity of the inner and outer layers, which was measured using a mercury injection pore measuring instrument, was found to be greater than 60%. When the scaffold was stretched to 400% of its original length, it returned to more than 98% of its original length.

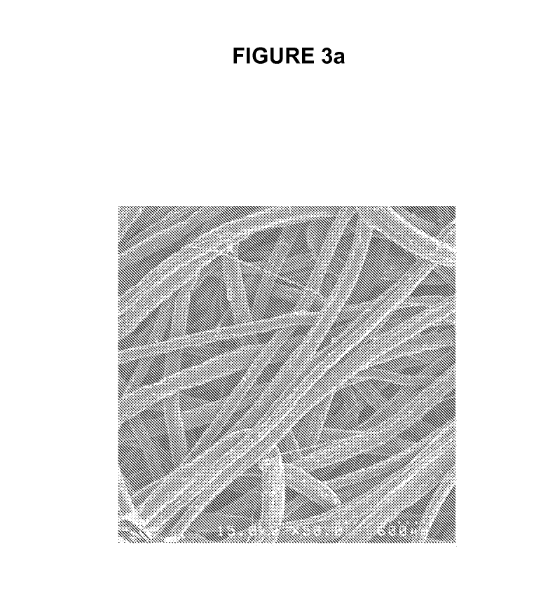

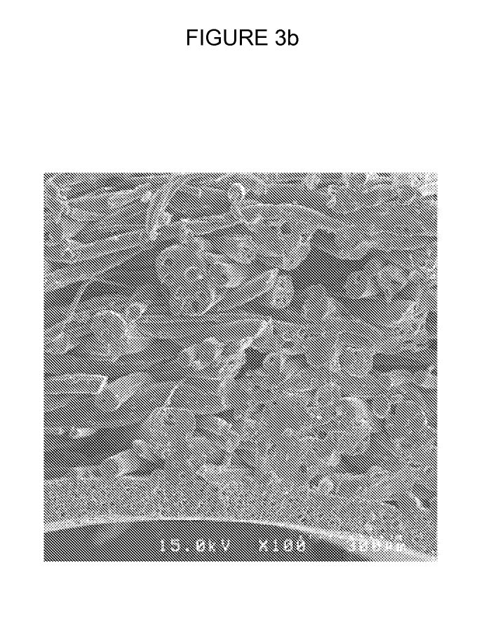

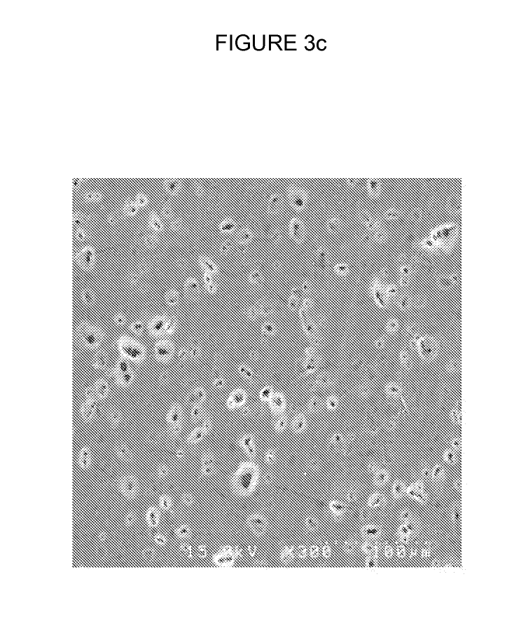

The porous scaffold was observed under a scanning electron microscope (SEM). The outer surface of the porous PLCL scaffold was found to have a fibrous structure (FIG. 3a). The inner surface of the PLCL scaffold contained few pores (FIG. 3c). A cross-sectional SEM micrograph of the porous PLCL scaffold revealed that the outer gel-phase polymer fibrous layer and the inner porous coating layer were properly attached to each other, and that the outer layer was highly interconnected between pores.

### Comparative Example 1

### Preparation of a Single-Layered Porous Scaffold

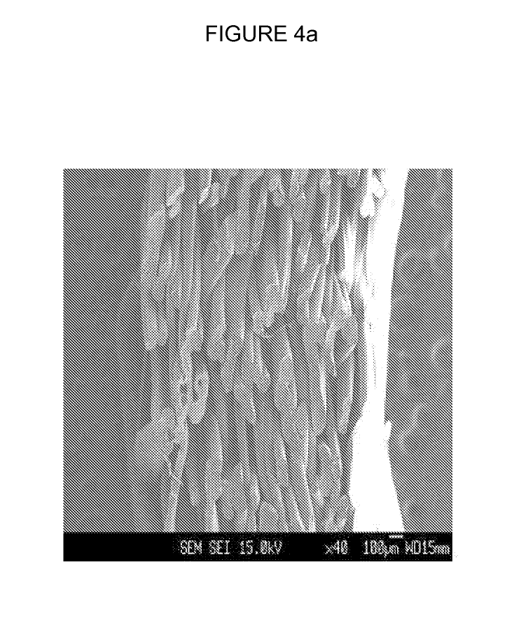

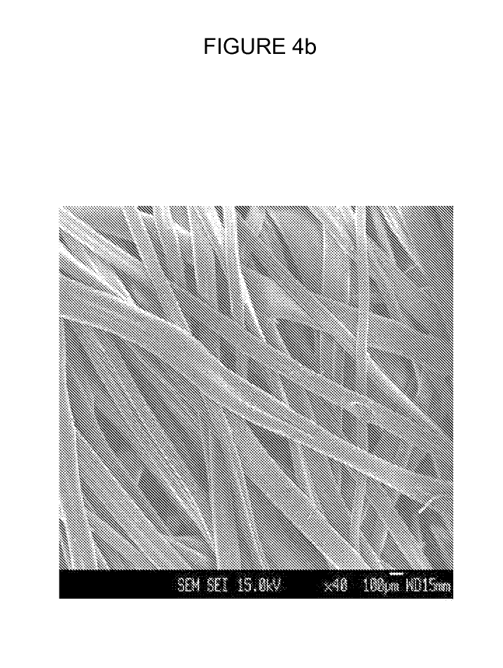

A single-layered porous scaffold was fabricated by gel spinning a highly viscous PLCL solution onto a rotating cylindrical shaft according to the same method as in Example 1, except that the cylindrical shaft was coated with the PLCL solution instead of the PLCL/NaCl mixture. The cross section and inner surface of the single-layered porous scaffold thus obtained were observed under a scanning electron microscope, and are shown in FIGS. 4a and 4b, respectively.

### Test Example 1

### Evaluation of Burst Strength and Blood Leakage

The double-layered porous scaffolds prepared in Example 1 were evaluated to estimate the burst strength thereof and the leakage of blood therefrom. A predetermined amount of human blood was put into a tube, which was connected to the porous scaffolds. Then, pneumatic pressure was slowly applied to the tube up to 1500 mmHg. During the pressure application, the pressure at which the scaffold was deformed and the blood leaked from the scaffold was recorded. The results are given in Table 1, below. The single-layered porous scaffold prepared in Comparative Example 1 was used as a comparative group.

TABLE 1

| | Burst pressure | Leakage pressure |
|---|---|---|
| | (mmHg) | (mmHg) |
| Single-layered scaffold | — | 30 |
| Double-layered scaffold | >1500 | >1500 |
| (PLCL/NaCl, 9:1) | | |
| Double-layered scaffold | 1200 | 1200 |
| (PLCL/NaCl, 2:1) | | |
| Double-layered scaffold | 1200 | 1200 |
| (PLCL/NaCl, 1:1) | | |

As shown in Table 1, in the case of the single-layered porous scaffold of Comparative Example 1, which did not have an inner porous coating layer, the blood leakage occurred at lower than 30 mmHg, and the burst pressure could not be measured because the burst pressure was lower than 30 mmHg. In contrast, the porous scaffold having a double-layered structure comprising an inner porous coating layer and an outer polymer coating layer, prepared in Example 1 according to the present method, did not exhibit deformation or leakage even at 1200 mmHg. In particular, the double-layered scaffold constructed at a PLCL to NaCl ratio of 9:1 did not burst even at higher than 1500 mmHg.

### Test Example 2

### Evaluation of Cell Seeding and Proliferation Efficiencies

The double-layered porous scaffold prepared in Example 1 was evaluated for cell seeding and proliferation efficiencies, as follows. Bone marrow was collected from the rump bone of a dog. Bone marrow mononuclear cells were isolated from the bone marrow using Ficoll density gradient separation. 1×10⁵ cells were seeded onto the porous scaffold. Then, the porous scaffold was implanted into the abdominal aorta of a dog. Thereafter, the scaffold was observed under a scanning electron microscope in order to determine whether cells were effectively grown into the micropores formed in the inner porous coating layer. The single-layered porous scaffold prepared in Comparative Example 1 was used as a comparative group.

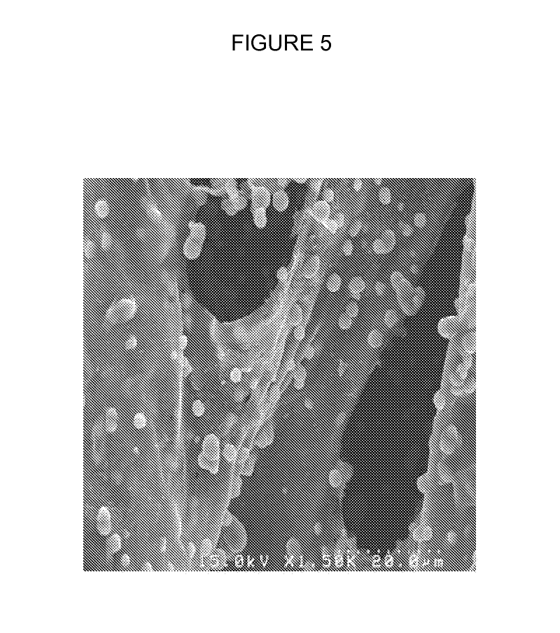

As shown in FIG. 5, bone marrow mononuclear cells were grown and proliferated in the pores formed in the inner porous coating layer of the double-layered porous scaffold according to the present invention. Also, this scaffold was found not to burst, to leak no blood, and to exhibit almost no clotting.

As described hereinbefore, unlike conventional porous scaffolds, the double-layered porous scaffold prepared according to the present method is well interconnected between pores, so that is able to effectively induce cell ingrowth and proliferation within pores, thereby being very advantageously used in three-dimensional tissue reconstruction. In particular, owing to its high mechanical strength and the microporosity of its inner layer, the double-layered porous scaffold effectively prevents early bursting and blood leakage even when implanted without in vitro cultivation. Thus, the double-layered porous scaffold may be useful as a biodegradable and biocompatible tissue-engineered artificial blood vessel.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A method of preparing a tube-type porous scaffold having a double-layered structure comprising the steps of:
(1) dissolving a biodegradable polymer in an organic solvent and mixing the polymer with a porogen to provide a polymer/porogen mixture;
(2) coating a cylindrical shaft with the polymer/porogen mixture so as to form an inner porous coating layer;
(3) preparing a biodegradable polymer gel by dissolving a biodegradable polymer in an organic solvent;
(4) spinning down the biodegradable polymer gel in a non-solvent coagulation bath in which the cylindrical shaft having the inner porous coating layer, obtained at step (2), is immersed and rotated to form gel-phase fibers and allowing the gel-phase fibers to wind around the inner porous coating layer of the rotating shaft so as to form an outer polymer fibrous layer; and
(5) separating the double-layered porous scaffold, formed on the shaft, from the shaft and removing the organic solvent and the porogen from the scaffold.

2. The method as set forth in claim 1, wherein the biodegradable polymer of step (1) is selected from the group consisting of poly(L-lactic acid) (PLLA), poly(D,L-lactic acid) (PDLLA), polyglycolic acid (PGA), polycaprolactone (PCL), polyhydroxyalkanoate, polydioxanone (PDS), polytrimethylene carbonate, and derivatives and copolymers thereof.

3. The method as set forth in claim 1, wherein the biodegradable polymer of step (1) has a molecular weight ranging from 5,000 to 1,000,000 Daltons.

4. The method as set forth in claim 1, wherein the biodegradable polymer of step (1) is dissolved in the organic solvent in an amount of 1% to 20% based on a weight to volume ratio (w/v) thereof.

5. The method as set forth in claim 1, wherein the organic solvent of step (1) is selected from the group consisting of chloroform, methylene chloride, acetic acid, ethylacetate, dimethylcarbonate, and tetrahydrofuran.

6. The method as set forth in claim 1, wherein the porogen of step (1) is selected from the group consisting of sodium chloride, sodium bicarbonate, ammonium bicarbonate, paraffin and polyethylene glycol.

7. The method as set forth in claim 1, wherein the porogen of step (1) is mixed with the biodegradable polymer in the biodegradable polymer solution at a polymer to porogen weight ratio ranging from 9:1 to 1:2.

8. The method as set forth in claim 1, wherein the coating of step (2) is carried out using a method selected from the group consisting of extrusion, impregnation, electrospinning, freeze-drying, phase separation, particle leaching, gas foaming, hydrocarbon templating, and melt molding.

9. The method as set forth in claim 1, wherein the inner porous coating layer of step (2) contains pores of less than 40 microns.

10. The method as set forth in claim 1, wherein the biodegradable polymer of step (3) is present in an amount of 4 to 20 wt % in the biodegradable polymer gel.

11. The method as set forth in claim 1, wherein, at step (4), the gel-phase fibers have a diameter ranging from 50 to 150 microns, and the outer polymer fibrous layer contains pores having a size ranging from 10 to 500 microns.

12. The method as set forth in claim 1, wherein the non-solvent of step (4) is selected from the group consisting of water, methanol, ethanol, butanol, hexane, heptane, and cyclohexane.

13. The method as set forth in claim 1, wherein the organic solvent and porogen of step (5) are removed through drying under reduced pressure and dissolution, respectively.

14. A tube-type porous scaffold for use as a biodegradable and biocompatible artificial blood vessel, which is prepared according to the method of claim 1 and has a double-layered structure comprising an inner porous coating layer and an outer polymer fibrous layer.

15. The tube-type porous scaffold as set forth in claim 14, wherein the inner porous coating layer has a pore size of less than 40 microns, and the outer polymer fibrous layer has a pore size of 10 to 500 microns.
